(19) United States
(12) Patent Application Publication
(10) Pub. No.: US 2024/0180588 A1
(43) Pub. Date: Jun. 6, 2024

(54) DISPOSABLE COMBINATION CAMERA BRIDGE AND INSTRUMENT CANNULA FOR SURGICAL APPLICATIONS

(71) Applicant: Central Access Solutions, LLC, Charlotte, NC (US)

(72) Inventor: Durham Weeks, Charlotte, NC (US)

(21) Appl. No.: 18/529,122

(22) Filed: Dec. 5, 2023

Related U.S. Application Data
(60) Provisional application No. 63/430,414, filed on Dec. 6, 2022.

(51) Int. Cl. A61B 17/34; A61B 90/00; A61M 39/06

(52) U.S. Cl. CPC A61B 17/3423; A61B 17/3417; A61B 17/3498; A61B 90/361; A61M 39/06; A61M 2039/0626; A61M 2205/02

(57) ABSTRACT

A minimally invasive surgery (MIS) cannula that is manufactured primarily from a plastic material such that it is disposable and that serves as both a camera bridge, to which a camera may be coupled, and an instrument or tool cannula or port, through which an instrument or tool may be used. The cannula optionally includes one or more ports to which an irrigating fluid line may be attached to enhance site visibility and access and a camera receptacle to which a camera can be affixed. When the camera is not used, a cap may be attached to the cannula using the camera receptacle and may include a valve or seal assembly or the like through which an instrument or tool may be used. The cap and valve or seal assembly or the like may also accommodate a camera and arthroscope attachment. Otherwise, the cap may simply seal the cannula.

### CROSS-REFERENCE TO RELATED APPLICATION

The present disclosure claims the benefit of priority of co-pending U.S. Provisional Patent Application No. 63/430,414, filed on Dec. 6, 2022, and entitled “DISPOSABLE COMBINATION CAMERA BRIDGE AND INSTRUMENT CANNULA FOR SURGICAL APPLICATIONS,” the contents of which are incorporated in full by reference herein.

### TECHNICAL FIELD

The present disclosure relates generally to the minimally invasive surgery (MIS) field. More particularly, the present disclosure relates to a disposable combination camera bridge and instrument cannula for surgical applications.

### BACKGROUND

In MIS procedures, it is common for a surgeon to visualize an area of interest using a camera coupled to a bridge disposed through the skin and musculature or a camera coupled to an arthroscope disposed through a cannula or port disposed through the skin and musculature. It is also common for the surgeon to manipulate the area of interest using instruments or tools disposed through a cannula or port disposed through the skin and musculature. Typically, these cannulas or ports include a dual valve or the like through which the arthroscope or instrument or tool is sealingly disposed. Caps may be used to seal the cannulas or ports when not in use.

Often, the surgeon has to place multiple bridges to accommodate multiple camera locations desired for a procedure, as well as multiple cannulas or ports to accommodate multiple instrument or tool locations, with each of these locations serving a distinct purpose. The bridges are usually manufactured from a metallic material and are sterilized between uses, while the cannulas or ports may be manufactured from a disposable plastic material or a sterilizable metallic material. In any event, the bridges and cannulas or ports are typically distinct components used for distinct purposes, which promotes inefficiency and results in unnecessary expense.

The present background is provided as illustrative environmental context only and should not be construed to be limiting in any manner. The concepts and principles of the present disclosure may be implemented in other contexts without limitation.

### SUMMARY

The present disclosure provides a MIS cannula that is manufactured primarily from a plastic material such that it is disposable and that serves as both a camera bridge, to which a camera may be coupled, and an instrument or tool cannula or port, through which an instrument or tool may be used. The cannula includes one or more ports to which an irrigating fluid line may be attached to enhance site visibility and access and a camera receptacle to which a camera can be affixed. When the camera is not used, a cap may be attached to the cannula using the camera receptacle and may include a valve or seal assembly or the like through which an instrument or tool may be used. The cap and valve or seal assembly or the like may also accommodate a camera and arthroscope attachment. Otherwise, the cap may simply seal the cannula. Thus, a versatile cannula is provided with a beneficial cap, such that the cannula may alternately act as a camera bridge, an access port, and a closure port.

As a result, disposable cannulas may be placed at several different locations and each used for various purposes. This promotes efficiency and reduces expense.

In one illustrative embodiment, the present disclosure provides a cannula assembly, including: an elongate cannula member; and an attachment structure coupled to an end portion of the elongate cannula member, wherein the attachment structure is adapted to securely receive either of a camera assembly and a cap structure. The elongate cannula member is manufactured from one or more of a metallic material, a plastic material, and a ceramic material. The cannula assembly also includes a tip member coupled to and disposed about another end portion of the elongate cannula member, wherein the tip member is manufactured from one or more of a metallic material, a plastic material, and a ceramic material that protects tissue from damage. The cannula assembly further includes a smooth, ribbed, threaded, or textured sheath structure coupled to and disposed about the end portion of the elongate cannula member, wherein the smooth, ribbed, threaded, or textured sheath structure is manufactured from one or more of a metallic material, a plastic material, and a ceramic material. Optionally, the cannula assembly includes one or more fluid ports coupled to the elongate cannula member. All fluid ports are on/off or adjustable. The attachment structure is manufactured from one or more of a metallic material, a plastic material, and a ceramic material. Optionally, the cap structure includes a valve or seal assembly and defines a port adapted to receive either of an instrument and an arthroscope. The attachment structure and the elongate cannula member are adapted to receive an obturator, trocar, or dilator device including an elongate member and a handle member. The obturator, trocar, or dilator device is manufactured from one or more of a metallic material, a plastic material, and a ceramic material. Optionally, the elongate member of the obturator, trocar, or dilator device includes a core member manufactured from a metallic material.

In another illustrative embodiment, the present disclosure provides a method for providing a cannula assembly, the method including: providing an elongate cannula member; and coupling an attachment structure to an end portion of the elongate cannula member, wherein the attachment structure is adapted to securely receive either of a camera assembly and a cap structure. The elongate cannula member is manufactured from one or more of a metallic material, a plastic material, and a ceramic material. The method also includes providing a tip member coupled to and disposed about another end portion of the elongate cannula member, wherein the tip member is manufactured from one or more of a metallic material, a plastic material, and a ceramic material. The method further includes providing a smooth, ribbed, threaded, or textured sheath structure coupled to and disposed about the end portion of the elongate cannula member, wherein the smooth, ribbed, threaded, or textured sheath structure is manufactured from one or more of a metallic material, a plastic material, and a ceramic material. Optionally, the method further includes providing one or more fluid ports coupled to the elongate cannula member. The attachment structure is manufactured from one or more of a metallic material, a plastic material, and a ceramic material. Optionally, the cap structure includes a valve or seal assembly and defines a port adapted to receive either of an instrument and an arthroscope. The attachment structure and the elongate cannula member are adapted to receive an obturator, trocar, or dilator device including an elongate member and a handle member. The obturator, trocar, or dilator device is manufactured from one or more of a metallic material, a plastic material, and a ceramic material. Optionally, the elongate member of the obturator, trocar, or dilator device includes a core member manufactured from a metallic material.

In a further illustrative embodiment, the present disclosure provides a cannula assembly, including: an elongate cannula member; and an attachment structure coupled to an end portion of the elongate cannula member, wherein the attachment structure is adapted to alternately receive: a camera assembly; a cap structure acting as a closure for the elongate cannula member; and the cap structure acting as a port for introducing one of an instrument and an arthroscope coupled to the camera assembly into the elongate cannula member.

In a still further illustrative embodiment, the present disclosure provides a method for using a cannula assembly, the method including: providing an elongate cannula member including an attachment structure coupled to an end portion of the elongate cannula member; and alternately coupling one of the following to the attachment structure: a camera assembly; a cap structure acting as a closure for the elongate cannula member; and the cap structure acting as a port for introducing one of an instrument and an arthroscope coupled to the camera assembly into the elongate cannula member.

### BRIEF DESCRIPTION OF THE DRAWINGS

The present disclosure is illustrated and described herein with reference to the various drawings, in which like reference numbers are used to denote like assembly components and method steps, as appropriate, and in which:

FIG. 1 illustrates a conventional sterilizable metallic camera bridge;

FIG. 2 illustrates a camera coupled to a conventional camera bridge;

FIG. 3 illustrates a camera coupled to a conventional arthroscope attachment;

FIG. 4 illustrates a camera coupled to a conventional arthroscope attachment used through a conventional cannula or port;

FIG. 5 illustrates a conventional disposable plastic cannula or port;

FIG. 6 illustrates a conventional sterilizable metallic cannula or port;

FIG. 7 illustrates conventional obturators and dilators used with conventional cannulas or ports;

FIG. 8 illustrates example instruments and tools;

FIG. 9 illustrates one embodiment of the cannula assembly of the present disclosure, including an associated obturator and cap assembly;

FIG. 10 further illustrates one embodiment of the cannula assembly of the present disclosure, including the associated obturator and cap assembly;

FIG. 11 illustrates one embodiment of the obturator of the present disclosure;

FIG. 12 illustrates one embodiment of the cannula assembly of the present disclosure coupled to an associated camera;

FIG. 13 further illustrates one embodiment of the cannula assembly of the present disclosure, including the associated cap assembly;

FIG. 14 further illustrates one embodiment of the cannula assembly of the present disclosure, highlighting an associated camera receptacle and fluid system connection;

FIG. 15 further illustrates one embodiment of the obturator of the present disclosure;

FIG. 16 further illustrates one embodiment of the cannula assembly of the present disclosure, including the associated cap assembly;

FIG. 17 further illustrates one embodiment of the cannula assembly of the present disclosure, including the associated obturator and cap assembly; and

FIG. 18 illustrates one embodiment of a method of use of the cannula assembly of the present disclosure in an example hip procedure.

### DETAILED DESCRIPTION

Again, the present disclosure provides a MIS cannula that is manufactured primarily from a plastic material such that it is disposable and that serves as both a camera bridge, to which a camera may be coupled, and an instrument or tool cannula or port, through which an instrument or tool may be used. The cannula includes one or more ports to which an irrigating fluid line may be attached to enhance site visibility and access and a camera receptacle to which a camera can be affixed. When the camera is not used, a cap may be attached to the cannula using the camera receptacle and may include a valve or seal assembly or the like through which an instrument or tool may be used. The cap and valve or seal assembly or the like may also accommodate a camera and arthroscope attachment. Otherwise, the cap may simply seal the cannula.

As a result, disposable cannulas may be placed at several different locations and each used for various purposes. This promotes efficiency and reduces expense.

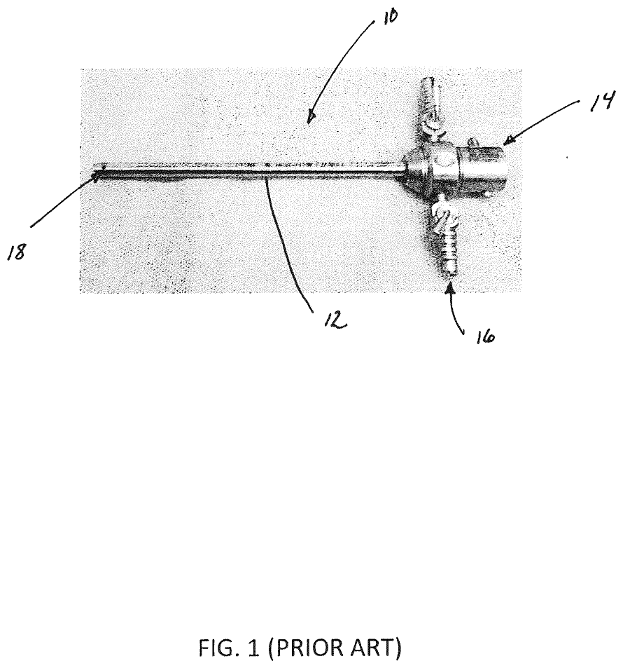

FIG. 1 illustrates a conventional sterilizable metallic camera bridge 10. The bridge 10 generally includes an elongate tubular member 12 that is adapted to be inserted through the skin and musculature of a patient to a site to be visualized and an attachment structure 14 that is adapted to receive and secure a camera to the elongate tubular member 12. Such attachment structures 14 typically conform to the type of camera to be attached, such that each bridge 10 is specific to a given type of camera. One or more fluid ports 16 are provided, to which a fluid inlet/outlet may be attached to deliver fluid to the site to be visualized. Further, a fluid outlet port 18 may be provided to allow fluid to exit the end of the elongate tubular member 12 adjacent to the site to be visualized. Such bridges 10 are typically manufactured from a metallic material and are sterilizable.

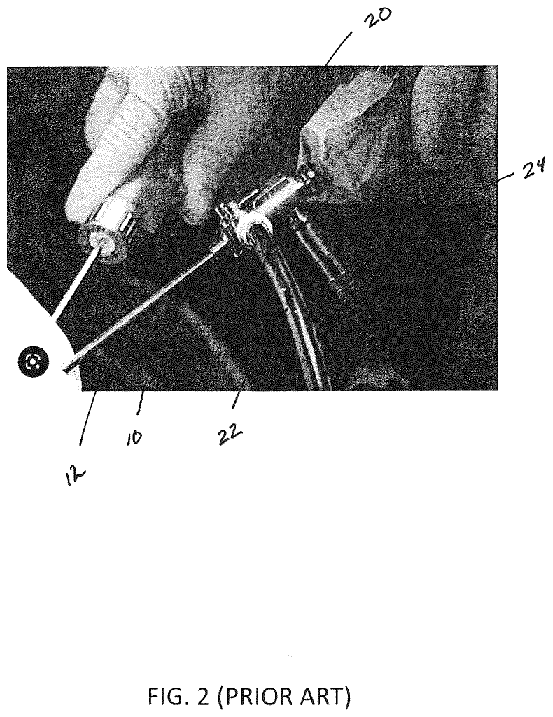

FIG. 2 illustrates a camera 20 coupled to a conventional camera bridge 10. A fluid inlet line 22 is coupled to the bridge 10, and a light source 24 is coupled to the camera 20. Light from the light source 24 is delivered to the site to be visualized through the elongate tubular member 12, as is fluid that aides in visualization.

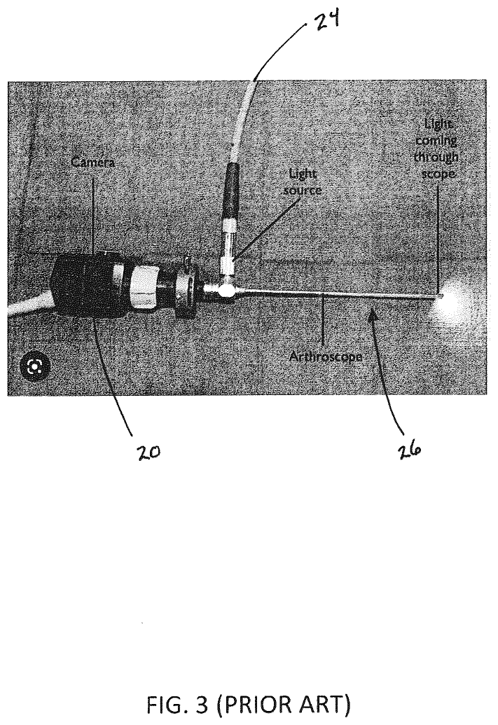

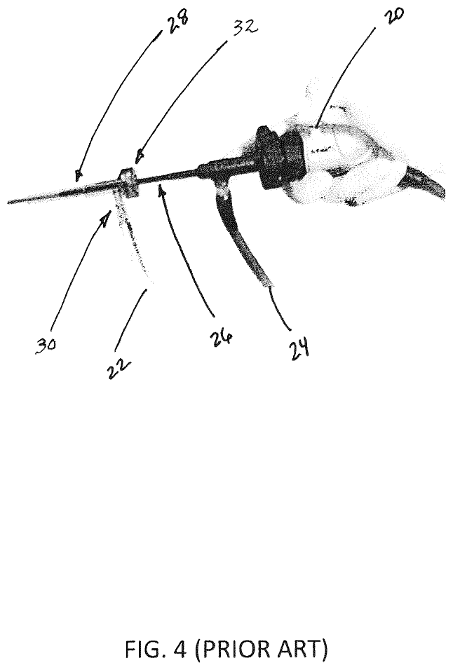

FIG. 3 illustrates a camera 20 coupled to a conventional arthroscope attachment 26. In this case, the arthroscope attachment 26 is typically inserted through a separate cannula or port 28, as is illustrated in FIG. 4. Light from the light source 24 is delivered to the site to be visualized through the arthroscope 26, while fluid that aides in visualization is delivered to the site through the separate cannula or port 28. The fluid is delivered to the cannula or port 28 through the associated fluid inlet port 30 and fluid inlet line 22. Here, the arthroscope attachment 26 is inserted through a dual valve cap structure 32 or the like coupled to the end of the cannula or port 28.

As mentioned herein above, the surgeon typically has to place multiple bridges to accommodate multiple camera locations desired for a procedure, as well as multiple cannulas or ports to accommodate multiple instrument or tool (or camera) locations, with each of these locations serving a distinct purpose. The bridges are usually manufactured from a metallic material and are sterilized between uses, while the cannulas or ports may be manufactured from a disposable plastic material or a sterilizable metallic material. In any event, the bridges and cannulas or ports are typically distinct components used for distinct purposes, which promotes inefficiency and results in unnecessary expense.

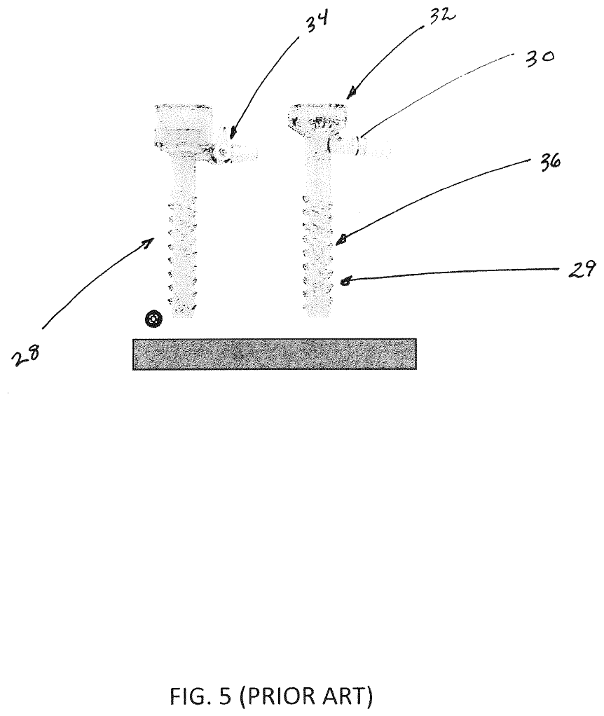

FIG. 5 illustrates a conventional disposable plastic cannula or port 28. The cannula or port 28 includes an elongate tubular member 29 that is adapted to be inserted through the skin and musculature of a patient to a site to be manipulated. The fluid inlet port 30 is in fluid communication with this elongate tubular member 29 and may include a suitable valve assembly 34 for permitting/preventing fluid flow. The dual valve cap assembly 32 is provided to seal the end of the cannula or port 28 and permit selective engagement of the instrument, tool, or camera and arthroscope. As illustrated a ribbed or textured structure 36 is provided around a portion of the elongate tubular member 29 to prevent the cannula or port 28 from backing out of the skin and musculature once placed. Here, the cannula or port is manufactured from a plastic material and is disposable, but the cannula or port 28 cannot serve as a bridge for the camera, as it does not include a suitable attachment mechanism for the camera.

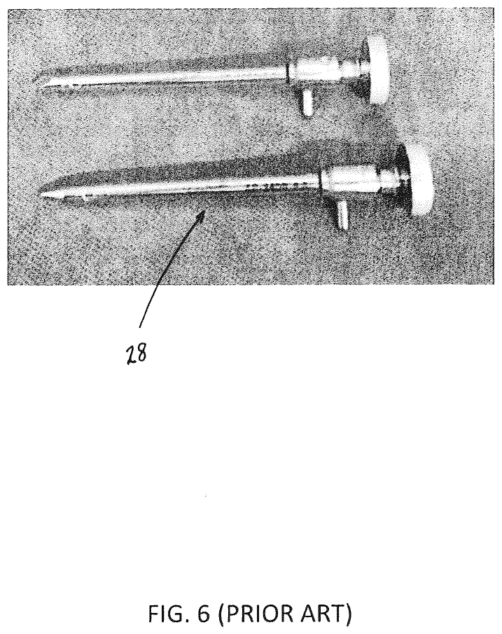

FIG. 6 illustrates a conventional sterilizable metallic cannula or port 28, which serves a similar purpose as the disposable plastic cannula or port 28 illustrated in FIG. 5. Again, the cannula or port 28 cannot serve as a bridge for the camera, as it does not include a suitable attachment mechanism for the camera.

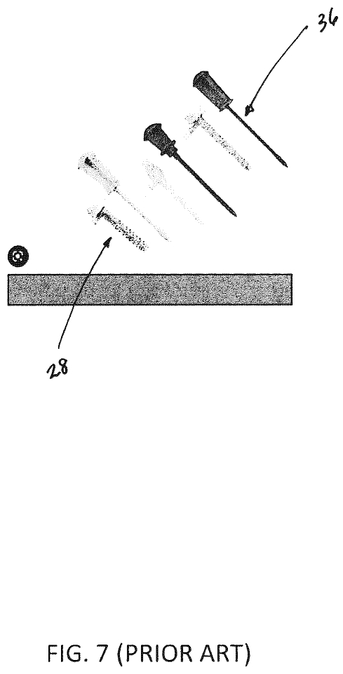

FIG. 7 illustrates conventional obturators and dilators 36 used with conventional cannulas or ports 28. In general, an obturator 36 is used to seal the piercing end of the cannula or port 28 upon placement, such that placement is made easier and less intrusive, while a dilator 36 is used to expand the placement area. Often, obturators and dilators 36 are inserted over guide wires or the like precisely placed at the site of interest under visualization.

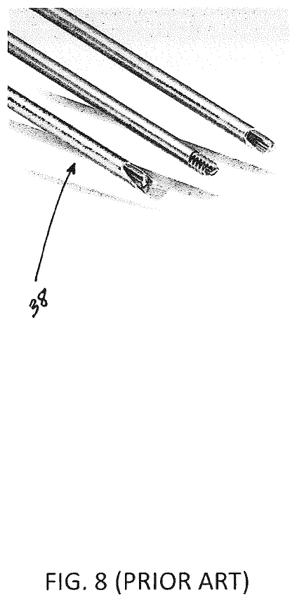

FIG. 8 illustrates example instruments and tools 38 used with the cannulas and ports 28 of FIGS. 5 and 6. Such instruments and tools 38 include, but are not limited to, shavers, cutters, burrs, rasps, suture passers, suture anchors, hammers, graspers, biters, scissors, etc.

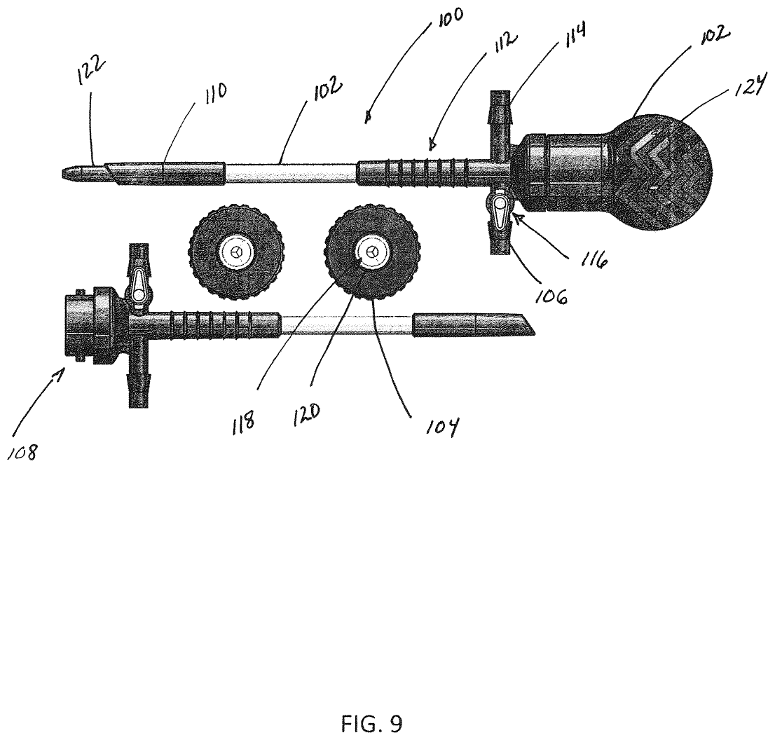

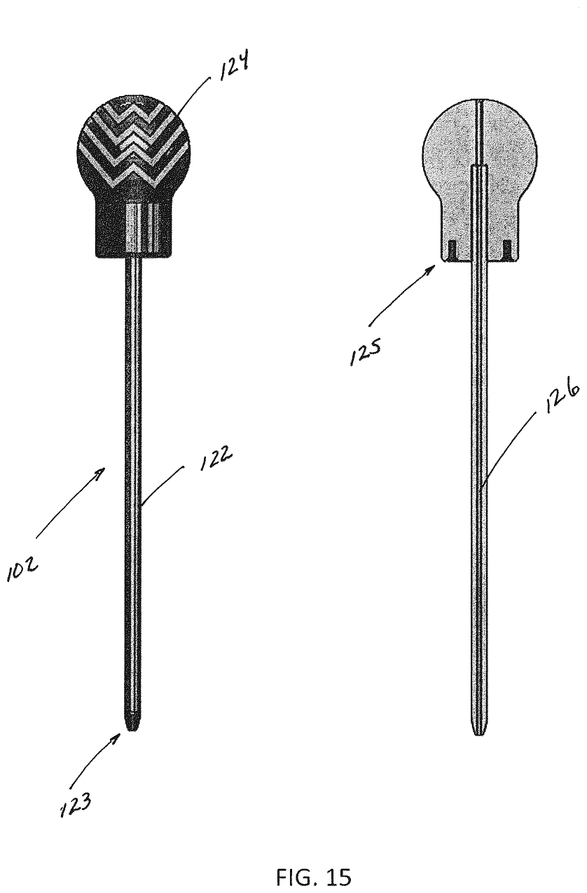

FIG. 9 illustrates one embodiment of the cannula assembly 100 of the present disclosure, including an associated obturator 102 and cap assembly 104. The cannula assembly 100 includes an elongate cannula member 102, a fluid inlet port 106 coupled to the elongate cannula member 102, and an attachment structure 108 coupled to an end portion of the elongate cannula member 102. The attachment structure 108 is adapted to securely receive either of a camera assembly and the cap structure 104, both of which preferably utilize the same fitting associated with the attachment structure 108. The elongate cannula member 102 is manufactured from one or more of a metallic material, a plastic material, and a ceramic material. The cannula assembly 100 also includes a tip member 110 coupled to and disposed about another end portion of the elongate cannula member 102. The tip member 110 is manufactured from one or more of a metallic material, a plastic material, and a ceramic material and prevents the site of interest from being exposed to any sharp edges of the elongate cannula member 102. The cannula assembly 100 further includes a ribbed or textured sheath structure 112 coupled to and disposed about the end portion of the elongate cannula member 102. The ribbed or textured sheath structure 112 is manufactured from one or more of a metallic material, a plastic material, and a ceramic material and serves to prevent the cannula assembly from backing out of the skin and musculature once placed. As illustrated, the cannula assembly still further includes another fluid outlet port 114 coupled to the elongate cannula member 102. Either or both of the fluid ports 106 and 114 may utilize conventional lure or other connections and may include a valve assembly 116 for selectively starting/stopping fluid flow there through. The attachment structure 108 is manufactured from one or more of a metallic material, a plastic material, and a ceramic material. As illustrated, the cap structure 104 includes a valve or seal assembly 118 and defines a port 120 adapted to receive either of an instrument or an arthroscope, or simply act as a sealing member. The attachment structure 108 and the elongate cannula member 102 are adapted to receive the obturator or dilator device 102 including an elongate member 122 that protrudes from the end of the elongate cannula member 102 and a handle member 124. The obturator or dilator device 102 is manufactured from one or more of a metallic material, a plastic material, and a ceramic material and is preferably securable to the attachment structure 108. Optionally, the elongate member 122 of the obturator or dilator device 102 includes a core member 126 manufactured from a metallic material, as is illustrated in FIG. 15.

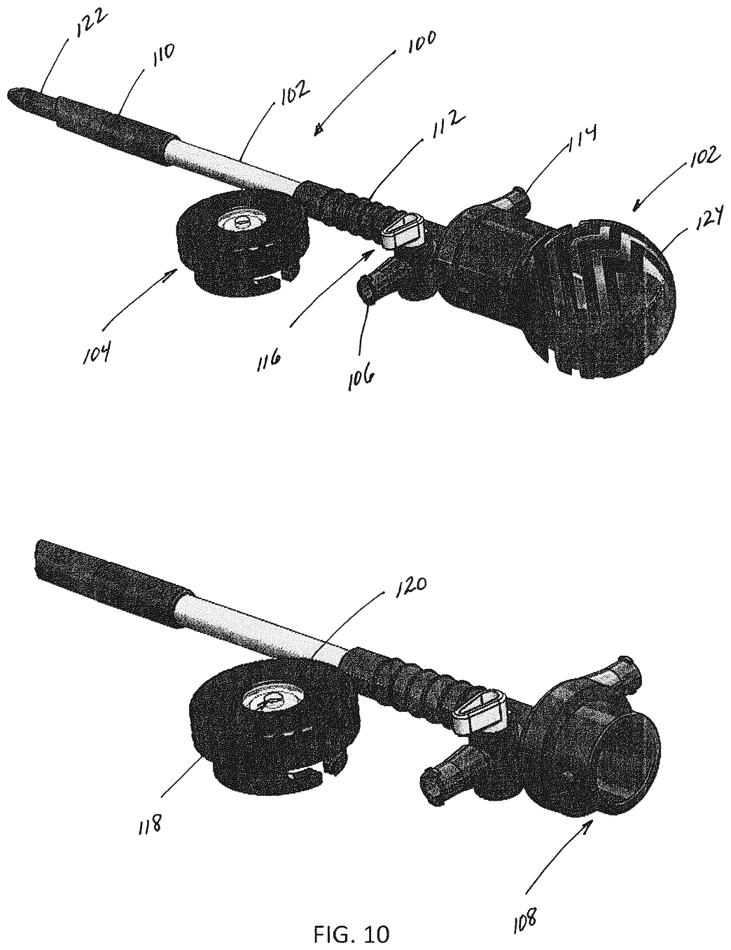

FIG. 10 further illustrates one embodiment of the cannula assembly 100 of the present disclosure, including an associated obturator 102 and cap assembly 104. The cannula assembly 100 includes an elongate cannula member 102, a fluid inlet port 106 coupled to the elongate cannula member 102, and an attachment structure 108 coupled to an end portion of the elongate cannula member 102. The attachment structure 108 is adapted to securely receive either of a camera assembly and the cap structure 104, both of which preferably utilize the same fitting associated with the attachment structure 108. The elongate cannula member 102 is manufactured from one or more of a metallic material, a plastic material, and a ceramic material. The cannula assembly 100 also includes a tip member 110 coupled to and disposed about another end portion of the elongate cannula member 102. The tip member 110 is manufactured from one or more of a metallic material, a plastic material, and a ceramic material and prevents the site of interest from being exposed to any sharp edges of the elongate cannula member 102. The cannula assembly 100 further includes a ribbed or textured sheath structure 112 coupled to and disposed about the end portion of the elongate cannula member 102. The ribbed or textured sheath structure 112 is manufactured from one or more of a metallic material, a plastic material, and a ceramic material and serves to prevent the cannula assembly from backing out of the skin and musculature once placed. As illustrated, the cannula assembly still further includes another fluid outlet port 114 coupled to the elongate cannula member 102. Either or both of the fluid ports 106 and 114 may utilize conventional lure or other connections and may include a valve assembly 116 for selectively starting/stopping fluid flow there through. The attachment structure 108 is manufactured from one or more of a metallic material, a plastic material, and a ceramic material. As illustrated, the cap structure 104 includes a valve or seal assembly 118 and defines a port 120 adapted to receive either of an instrument or an arthroscope, or simply act as a sealing member. The attachment structure 108 and the elongate cannula member 102 are adapted to receive the obturator or dilator device 102 including an elongate member 122 that protrudes from the end of the elongate cannula member 102 and a handle member 124. The obturator or dilator device 102 is manufactured from one or more of a metallic material, a plastic material, and a ceramic material and is preferably securable to the attachment structure 108. Optionally, the elongate member 122 of the obturator or dilator device 102 includes a core member 126 manufactured from a metallic material, as is illustrated in FIG. 15.

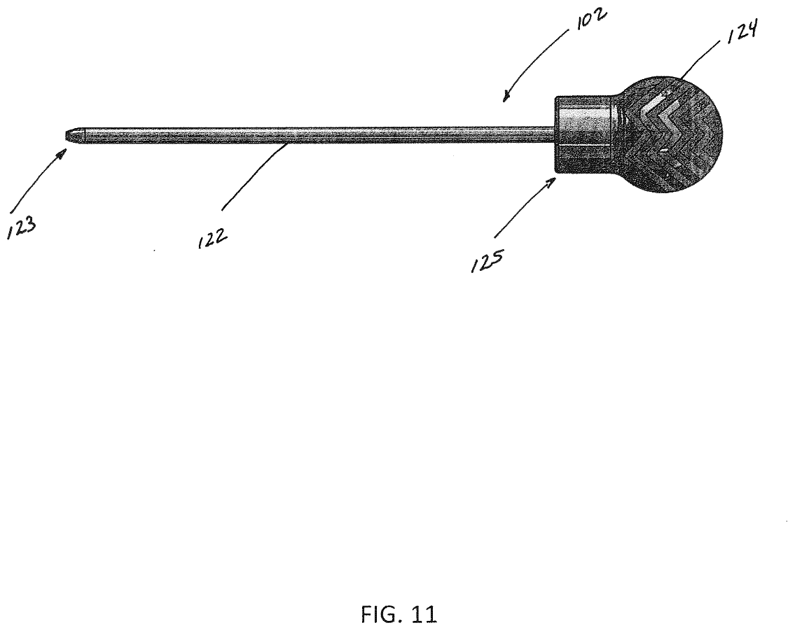

FIG. 11 illustrates one embodiment of the obturator 102 of the present disclosure. The obturator 102 is manufactured from one or more of a metallic material, a plastic material, and a ceramic material and is preferably securable to the attachment structure via a fitting adjacent to the handle 124. Again, the elongate member 122 of the obturator 102 optionally includes a core member 126 manufactured from a metallic material, as is illustrated in FIG. 15 to aide in visualization. As is illustrated, the end of the obturator 102 preferably includes a tapered tip 123 for engaging the site of interest.

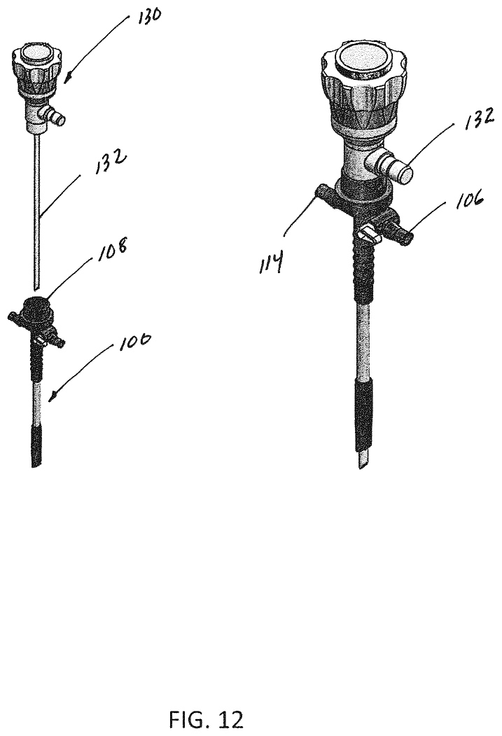

FIG. 12 illustrates one embodiment of the cannula assembly 100 of the present disclosure coupled to an associated camera 130. Specifically, the camera 130 securely engages the attachment structure 108 of the cannula assembly 100, with the arthroscope 132 of the camera 130 disposed concentrically within the elongate portion of the cannula assembly 100. The fluid inlet port 106 and fluid outlet port 114 associated with the cannula assembly 100 and the light source coupler 132 associated with the camera 130 are highlighted.

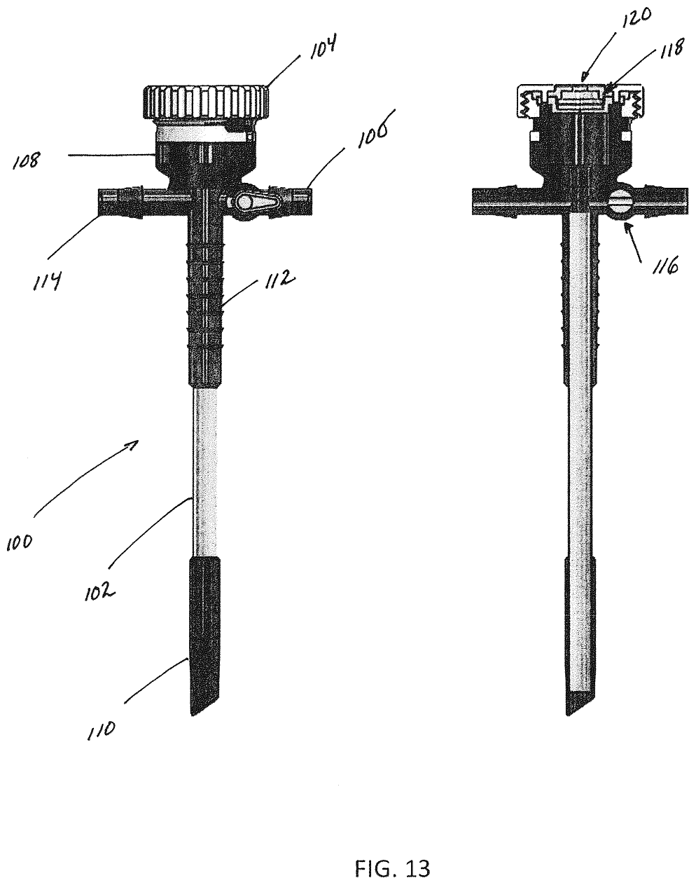

FIG. 13 further illustrates one embodiment of the cannula assembly 100 of the present disclosure, including the associated cap assembly 104. The cannula assembly 100 includes an elongate cannula member 102, a fluid inlet port 106 coupled to the elongate cannula member 102, and an attachment structure 108 coupled to an end portion of the elongate cannula member 102. The attachment structure 108 is adapted to securely receive either of a camera assembly and the cap structure 104, both of which preferably utilize the same fitting associated with the attachment structure 108. The elongate cannula member 102 is manufactured from one or more of a metallic material, a plastic material, and a ceramic material. The cannula assembly 100 also includes a tip member 110 coupled to and disposed about another end portion of the elongate cannula member 102. The tip member 110 is manufactured from one or more of a metallic material, a plastic material, and a ceramic material and prevents the site of interest from being exposed to any sharp edges of the elongate cannula member 102. The cannula assembly 100 further includes a ribbed or textured sheath structure 112 coupled to and disposed about the end portion of the elongate cannula member 102. The ribbed or textured sheath structure 112 is manufactured from one or more of a metallic material, a plastic material, and a ceramic material and serves to prevent the cannula assembly from backing out of the skin and musculature once placed. As illustrated, the cannula assembly still further includes another fluid outlet port 114 coupled to the elongate cannula member 102. Either or both of the fluid ports 106 and 114 may utilize conventional lure or other connections and may include a valve assembly 116 for selectively starting/stopping fluid flow there through. The attachment structure 108 is manufactured from one or more of a metallic material, a plastic material, and a ceramic material. As illustrated, the cap structure 104 includes a valve or seal assembly 118 and defines a port 120 adapted to receive either of an instrument or an arthroscope, or simply act as a sealing member.

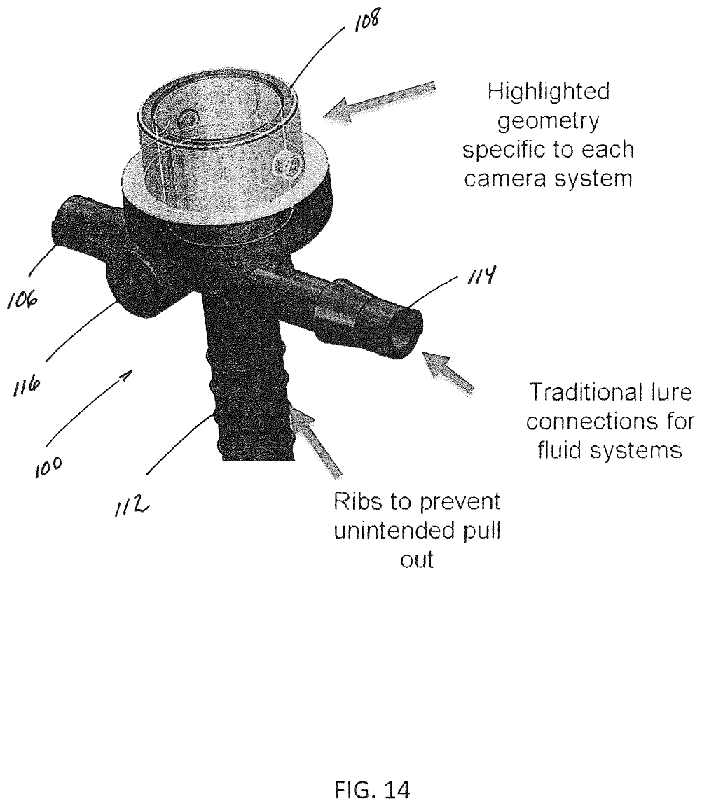

FIG. 14 further illustrates one embodiment of the cannula assembly 100 of the present disclosure, highlighting an associated camera receptacle 108 and fluid system connection. The cannula assembly 100 includes the fluid inlet port 106 and the attachment structure 108. The attachment structure 108 is adapted to securely receive the camera assembly and the cap structure, both of which preferably utilize the same fitting associated with the attachment structure 108. The ribbed or textured sheath structure 112 is manufactured from one or more of a metallic material, a plastic material, and a ceramic material and serves to prevent the cannula assembly from backing out of the skin and musculature once placed. As illustrated, the cannula assembly still further includes another fluid outlet port 114 coupled to the elongate cannula member 102. Either or both of the fluid ports 106 and 114 may utilize conventional lure or other connections and may include a valve assembly 116 for selectively starting/stopping fluid flow there through. The attachment structure 108 is manufactured from one or more of a metallic material, a plastic material, and a ceramic material.

FIG. 15 further illustrates one embodiment of the obturator 102 of the present disclosure. The obturator 102 is manufactured from one or more of a metallic material, a plastic material, and a ceramic material and is preferably securable to the attachment structure via a fitting 125 adjacent to the handle 124. Again, the elongate member 122 of the obturator 102 optionally includes a core member 126 manufactured from a metallic material to aide in visualization. As is illustrated, the end of the obturator 102 preferably includes a tapered tip 123 for engaging the site of interest.

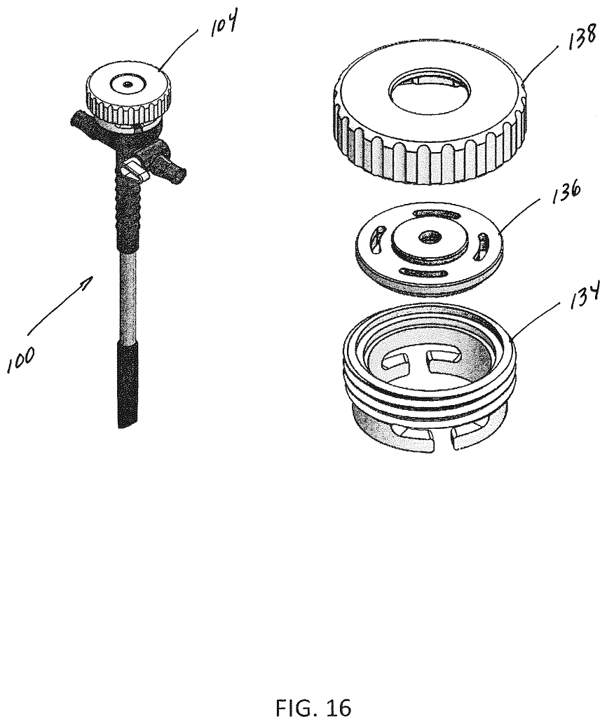

FIG. 16 further illustrates one embodiment of the cannula assembly 100 of the present disclosure, including the associated cap assembly 104. The cap assembly 104 includes a fitting 134 corresponding to the attachment structure of the cannula assembly 100, the valve or seal assembly 136 or the like, and a top member 138 that may be grasped and rotated by a user.

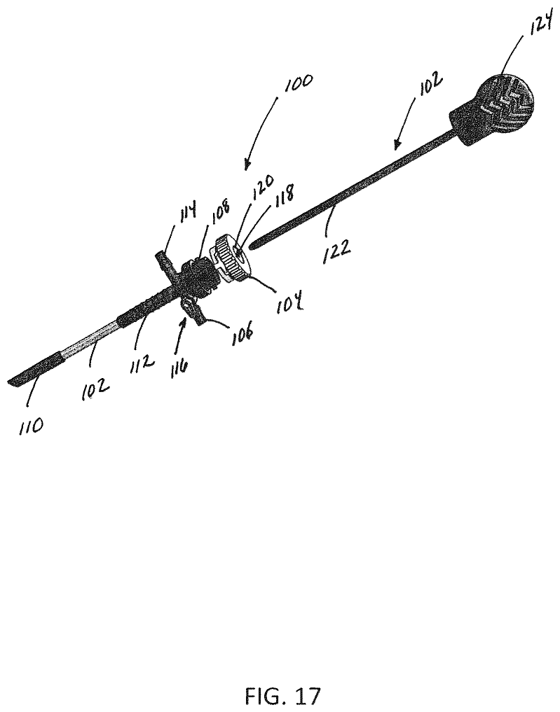

FIG. 17 further illustrates one embodiment of the cannula assembly 100 of the present disclosure, including the associated obturator 102 and cap assembly 104. The cannula assembly 100 includes an elongate cannula member 102, a fluid inlet port 106 coupled to the elongate cannula member 102, and an attachment structure 108 coupled to an end portion of the elongate cannula member 102. The attachment structure 108 is adapted to securely receive either of a camera assembly and the cap structure 104, both of which preferably utilize the same fitting associated with the attachment structure 108. The elongate cannula member 102 is manufactured from one or more of a metallic material, a plastic material, and a ceramic material. The cannula assembly 100 also includes a tip member 110 coupled to and disposed about another end portion of the elongate cannula member 102. The tip member 110 is manufactured from one or more of a metallic material, a plastic material, and a ceramic material and prevents the site of interest from being exposed to any sharp edges of the elongate cannula member 102. The cannula assembly 100 further includes a ribbed or textured sheath structure 112 coupled to and disposed about the end portion of the elongate cannula member 102. The ribbed or textured sheath structure 112 is manufactured from one or more of a metallic material, a plastic material, and a ceramic material and serves to prevent the cannula assembly from backing out of the skin and musculature once placed. As illustrated, the cannula assembly still further includes another fluid outlet port 114 coupled to the elongate cannula member 102. Either or both of the fluid ports 106 and 114 may utilize conventional lure or other connections and may include a valve assembly 116 for selectively starting/stopping fluid flow there through. The attachment structure 108 is manufactured from one or more of a metallic material, a plastic material, and a ceramic material. As illustrated, the cap structure 104 includes a valve or seal assembly 118 and defines a port 120 adapted to receive either of an instrument or an arthroscope, or simply act as a sealing member. The attachment structure 108 and the elongate cannula member 102 are adapted to receive the obturator or dilator device 102 including an elongate member 122 that protrudes from the end of the elongate cannula member 102 and a handle member 124. The obturator or dilator device 102 is manufactured from one or more of a metallic material, a plastic material, and a ceramic material and is preferably securable to the attachment structure 108. Optionally, the elongate member 122 of the obturator or dilator device 102 includes a core member 126 manufactured from a metallic material, as is illustrated in FIG. 15.

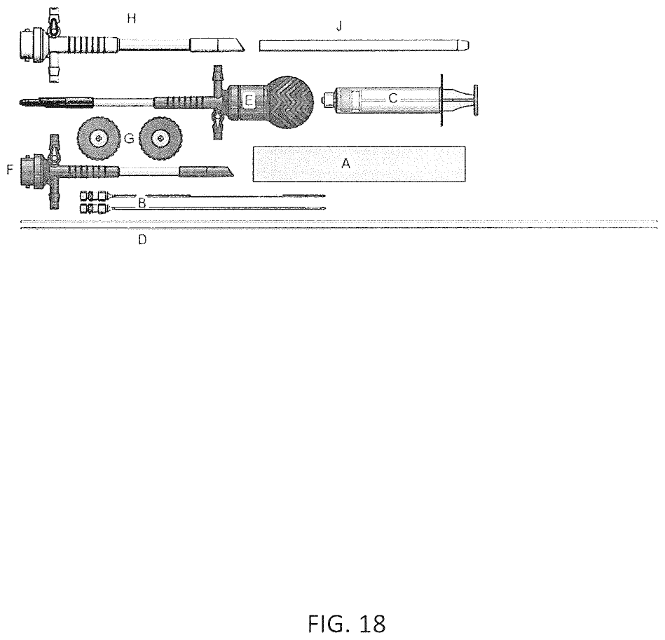

FIG. 18 illustrates one embodiment of a method of use of the cannula assembly of the present disclosure in an example hip procedure. Referring to the reference letters provided in the drawing:
A. Ruler: The ruler is first used to mark out the anatomic landmarks on the hip before introducing the arthroscopic needles;
B. Arthroscopic Needles: The arthroscopic needles are next used, under fluoroscopy, to safely access the central compartment of the hip. The inner trocar is then removed to allow next steps
C. Syringe: The syringe is next used to inject sterile saline into the joint to make visualization under arthroscopic imaging easier;
D. Nitinol Guide Wire: The nitinol guide wire is next used and delivered down the inner cannulation of the arthroscopic needle, the arthroscopic needle is then removed, leaving the nitinol guide wire in place;
E. First Cannula and Trocar: The first cannula and trocar (obturator) are next introduced over the nitinol guide wire into the central compartment, creating the lateral portal;
F. Second Cannula: The second cannula and trocar (obturator) from the first cannula are next used to repeat step E, creating the anterior lateral portal;
G. Cannula Cap: The cannula cap is next used once the portals (lateral and anterior lateral) have been established to create a fluid seal on the end of the cannulas;
H. Accessory Cannula: The accessory cannula is next used if a surgeon requires a cannula for the peripheral compartment in order to complete bony work on the head/neck area of the femur; and
J. Trocar Extender for the Accessory Cannula: The trocar extender for the accessory cannula is next used with the obturator or trocar from the cannula in step E, allowing the surgeon to place the trocar extender over the obturator and effectively fill a larger cannulation in the accessory cannula.

Although the present disclosure is illustrated and described herein with reference to illustrative embodiments and specific examples thereof, it will be readily apparent to those of ordinary skill in the art that other embodiments and examples may perform similar functions and/or achieve like results. All such equivalent embodiments and examples are within the spirit and scope of the present disclosure, are contemplated thereby, and are intended to be covered by the following non-limiting claims for all purposes.

1. A cannula assembly, comprising:
an elongate cannula member; and
an attachment structure coupled to an end portion of the elongate cannula member, wherein the attachment structure is adapted to securely receive either of a camera assembly and a cap structure.

2. The cannula assembly of claim 1, wherein the elongate cannula member is manufactured from one or more of a metallic material, a plastic material, and a ceramic material.

3. The cannula assembly of claim 1, further comprising a tip member coupled to and disposed about another end portion of the elongate cannula member, wherein the tip member is manufactured from one or more of a metallic material, a plastic material, and a ceramic material.

4. The cannula assembly of claim 1, further comprising a smooth, ribbed, threaded, or textured sheath structure coupled to and disposed about the end portion of the elongate cannula member, wherein the smooth, ribbed, threaded, or textured sheath structure is manufactured from one or more of a metallic material, a plastic material, and a ceramic material.

5. The cannula assembly of claim 1, further comprising one or more fluid ports coupled to the elongate cannula member.

6. The cannula assembly of claim 1, wherein the attachment structure is manufactured from one or more of a metallic material, a plastic material, and a ceramic material.

7. The cannula assembly of claim 1, wherein the cap structure comprises a valve or seal assembly and defines a port adapted to receive either of an instrument and an arthroscope.

8. The cannula assembly of claim 1, wherein the attachment structure and the elongate cannula member are adapted to receive an obturator, trocar, or dilator device comprising an elongate member and a handle member.

9. The cannula assembly of claim 8, wherein the obturator, trocar, or dilator device is manufactured from one or more of a metallic material, a plastic material, and a ceramic material.

10. The cannula assembly of claim 8, wherein the elongate member of the obturator, trocar, or dilator device comprises a core member manufactured from a metallic material.

11. A method for providing a cannula assembly, the method comprising:
providing an elongate cannula member; and
coupling an attachment structure to an end portion of the elongate cannula member, wherein the attachment structure is adapted to securely receive either of a camera assembly and a cap structure.

12. The method of claim 11, wherein the elongate cannula member is manufactured from one or more of a metallic material, a plastic material, and a ceramic material.

13. The method of claim 11, further comprising providing a tip member coupled to and disposed about another end portion of the elongate cannula member, wherein the tip member is manufactured from one or more of a metallic material, a plastic material, and a ceramic material.

14. The method of claim 11, further comprising providing a smooth, ribbed, threaded, or textured sheath structure coupled to and disposed about the end portion of the elongate cannula member, wherein the smooth, ribbed, threaded, or textured sheath structure is manufactured from one or more of a metallic material, a plastic material, and a ceramic material.

15. The method of claim 11, further comprising providing one or more fluid ports coupled to the elongate cannula member.

16. The method of claim 11, wherein the attachment structure is manufactured from one or more of a metallic material, a plastic material, and a ceramic material.

17. The method of claim 11, wherein the cap structure comprises a valve or seal assembly and defines a port adapted to receive either of an instrument and an arthroscope.

18. The method of claim 11, wherein the attachment structure and the elongate cannula member are adapted to receive an obturator, trocar, or dilator device comprising an elongate member and a handle member.

19. The method of claim 18, wherein the obturator, trocar, or dilator device is manufactured from one or more of a metallic material, a plastic material, and a ceramic material.

20. The method of claim 18, wherein the elongate member of the obturator, trocar, or dilator device comprises a core member manufactured from a metallic material.

21. A cannula assembly, comprising:
an elongate cannula member; and
an attachment structure coupled to an end portion of the elongate cannula member, wherein the attachment structure is adapted to alternately receive:
a camera assembly;
a cap structure acting as a closure for the elongate cannula member; and
the cap structure acting as a port for introducing one of an instrument and an arthroscope coupled to the camera assembly into the elongate cannula member.

22. A method for using a cannula assembly, the method comprising:
providing an elongate cannula member comprising an attachment structure coupled to an end portion of the elongate cannula member; and
alternately coupling one of the following to the attachment structure:
a camera assembly;
a cap structure acting as a closure for the elongate cannula member; and
the cap structure acting as a port for introducing one of an instrument and an arthroscope coupled to the camera assembly into the elongate cannula member.
